(19) United States
(12) Patent Application Publication
(10) Pub. No.: US 2007/0167527 A1
(43) Pub. Date: Jul. 19, 2007

(54) MEMANTINE FOR THE NORMALIZATION OF VISUAL ACUITY DEFICITS

(76) Inventors: James A. Burke, Santa Ana, CA (US); Kai-Ming Zhang, Irvine, CA (US)

Correspondence Address: Robert J. Baran (T2-7H);ALLERGAN, INC., Legal Department, 2525 Dupont Drive, Irvine, CA, 92612, US

(21) Appl. No.: 11/563,474

(22) Filed: Nov. 27, 2006

Related U.S. Application Data
(60) Provisional application No. 60/759,181, filed on Jan. 13, 2006.

(51) Int. Cl. A61K 31/13

(52) U.S. Cl. 514/662

(57) ABSTRACT

A method for the treatment of disturbance of visual function which comprises administering a pharmaceutically effective amount of memantine to a patient in need of treatment of said disturbance.

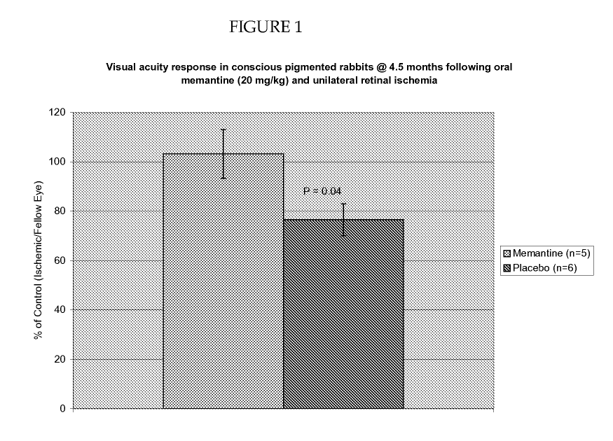

### CROSS REFERENCE TO RELATED APPLICATIONS

This application is based on, and claims the benefit of, U.S. Provisional Application No. 60/759,181, filed Jan. 13, 2006, and which is incorporated herein by reference.

### BACKGROUND OF THE INVENTION

1. Field of the Invention

The present invention relates to an agent for the prophylaxis and treatment of disturbance of visual function, e.g. by faulty refraction of light rays in the eye. By disturbance of visual function is meant a condition where normal vision cannot be obtained, i.e. ametropia, which condition is inclusive of myopia, hypermetropia, strabismus, disorders of the retina (which is a receptor of light), the condition where normal vision of an object is temporarily prevented by systemic or local fatigue of eyes, and other conditions, which disturb visual function.

2. Background of the Art

The determination of visual acuity is an essential part of every eye examination. During the course of such an examination, acuity may be measured repeatedly to ascertain the resolution of each eye independently and both eyes together. The examination may also consist of independent and combined testing of the eyes with the aid of corrective lenses. In fact, the repeated determination of acuity forms an essential part of the process of refracting or determining the optimal corrective lenses to alleviate the effects of ametropia as well as a means for assessing the progress of ocular pathology.

The retina consists of photoreceptor cells, bipolar cells, ganglion cells, horizontal cells, amacrine cells and the like, which transmit optical information to the central nerves. The functions of these cells contribute to the fulfillment of retinal function to organize the received optical information and transmit same to the central nerves.

When the retina is damaged, visual loss, disturbance of light sense and disturbance of visual field are induced, thereby causing central retinal artery and vein occlusions, congenital stationary night blindness, diabetic retinopathy, pigmentary retinal degeneration, retinal detachment, uveitis and the like.

While the therapeutics of retinal diseases and convalescence thereof vary depending on the kind and degree of the diseases, in particular, central retinal artery occlusion, diabetic retinopathy and retinal detachment scarcely allow complete recovery of retinal functions. It may happen that visual acuity does not improve after all and even an operation does not result in full recovery of visual acuity. What is more, no effective cure is currently available for pigmentary retinal degeneration but a symptomatic therapy such as use of sun glasses to avoid direct sun light.

Of the disturbances of visual function, myopia and hypermetropia refer to the condition wherein the light that passed through cornea cannot form an image on the retina, and thus cannot grasp the image clearly. Of these, myopia is divided into axial myopia and refractive myopia according to the cause of the condition. The refractive myopia is caused by an increased refraction of cornea and lens, while axial myopia is caused by an elongation of the eyeball in the direction of optic axis, i.e. axial direction.

In most cases, myopia is treated by a means utilizing correction of optical refraction. Correction of optical refraction by wearing glasses is not an ideal means as far as the quality of life and convenience for studying etc. are concerned. Correction of optical refraction using contact lenses often causes complications, and corneal ulcer may occur, which could possibly lead to the loss of sight in severe cases. In addition, recent application of corneal surgery to cure myopia is sometimes associated with failure to achieve expected levels of refraction, as well as occurrence of pain during operation and postoperative corneal opacity. In view of the fact that the correction of optical refraction and surgical operation such as the above-mentioned cannot be a perfect cure of myopia, treatment of myopia by the use of a drug is desired.

The asthenopia refers to a condition involving a kind of accommodation disorder of ciliary muscle due to systemic or local fatigue of the eye. Fatigue of eyes results in progressively growing distance of near point, which proceeds to the point that the eyes cannot recognize an object temporarily. However, recovery from fatigue can restore the original condition.

The treatment of asthenopia has heretofore included administration of medicaments such as vitamins (e.g., vitamin B.sub.1 and vitamin B.sub.12), ATP and the like, though sufficient therapeutic effects against asthenopia have not been attained.

Various recent patents have disclosed the use of memantine for treatment of various diseases and conditions of the eye. (See, for example, U.S. Pat. Nos. 5,597,809; 5,922,773 and 6,573,280.)

### DESCRIPTION OF THE DRAWING FIGURE

The FIGURE shows the visual acuity of rabbits treated with memantine or a placebo after a period of having elevated intraocular pressure (IOP).

### SUMMARY OF THE INVENTION

The present invention provides a method for the treatment of disturbance of visual function which comprises administering a pharmaceutically effective amount of memantine to a patient in need of treatment of said disturbance. Preferably, the method of this invention comprises local administration of memantine or a salt thereof to the eye.

For example, memantine may be administered in the form of a liquid preparation, i.e. memantine or a salt thereof may be administered in the form of an ophthalmic solution.

Alternatively, the method of this invention may comprise administering memantine or a salt thereof in the form of an injection, e.g. memantine or a salt thereof may be administered in the presence of a solubilizer.

The pharmaceutical preparation utilized in the method of the present invention may contain memantine or a salt thereof in a concentration of 0.01-2.0 (W/V) %.

The method of the present invention may be utilized to treat a disturbance of visual function resulting from an injury to the outer retina thereby causing visual acuity deficits.

Preferably, a patient treated according to the method of the present invention an improvement in reading a Snellen Eye Chart of at least one line.

### DETAILED DESCRIPTION OF THE INVENTION

Memantine can be also used alone, and after being converted to a pharmacologically acceptable salt. Examples of such salt include acid addition salts with inorganic acid, organic acid, acidic amino acids and the like.

The inorganic acid is exemplified by hydrochloric acid, hydrobromic acid, nitric acid, sulfuric acid, phosphoric acid and the like.

The organic acid is exemplified by formic acid, acetic acid, trifluoroacetic acid, fumaric acid, oxalic acid, tartaric acid, maleic acid, citric acid, succinic acid, malic acid, methanesulfonic acid, benzenesulfonic acid, p-toluenesulfonic acid, and the like.

The acidic amino acid is exemplified by aspartic acid and glutamic acid.

Memantine and a salt thereof have superior therapeutic effect on asthenopia, suppressive action on axial elongation, suppressive action on degradation of retinal functions, and retinal function-recovery action. Therefore, they are useful as agents for the prophylaxis and treatment of disturbance of visual functions.

The agents for the prophylaxis and treatment of disturbance of visual function of the present invention have low toxicity and can be administered safely to mammals such as human, rabbit, dog, cat, cow, horse, monkey and the like by an oral or parenteral route.

The agents for the prophylaxis and treatment of disturbance of visual function of the present invention can be produced by, for example, admixing memantine or a salt thereof with a pharmaceutically acceptable carrier.

The pharmaceutically acceptable carrier includes, for example, various organic and inorganic carriers commonly used as materials for preparations, such as, for solid preparations, excipients, lubricants, binders, disintegrators and the like, and, for liquid preparations, solvents, solubilizers, suspending agents, tackifiers, isotonizing agents, buffers, analgesic agents and the like, which can be used as appropriate. Where necessary, preservatives, chelating agents, antioxidants, colorings, sweeteners, flavors, aromatics, and other additives for preparations may be added by a conventional method.

Examples of suitable excipients include lactose, sucrose, mannitol, starch, crystalline cellulose, light anhydrous silicic acid and the like.

Examples of suitable solvents include water for injection, alcohols (e.g., ethanol, propylene glycol, macrogol, glycerine and the like), fats and oils (e.g., olive oil, sesame oil, peanut oil, cotton seed oil, castor oil, corn oil and the like), and the like.

Examples of suitable solubilizers include polyvinylpyrrolidone, cyclodextrin, caffeine, polyethylene glycol, propylene glycol, mannitol, benzyl benzoate, ethanol, trisaminomethane, cholesterol, triethanolamine, sodium carbonate, sodium citrate and the like.

Examples of suitable isotonizing agents include sorbitol, glycerol, polyethylene glycol, propylene glycol, glucose, sodium chloride and the like.

Examples of suitable buffers include phosphate buffer, borate buffer, citrate buffer, tartrate buffer, acetate buffer and the like.

Examples of suitable preservatives include p-hydroxybenzoate, benzalkonium chloride, benzethonium chloride, chlorobutanol, benzyl alcohol, phenethyl alcohol, dehydroacetic acid, sorbic acid and salt thereof, p-chlorometaxylenol, chlorocresol, thimerosal and the like.

Examples of suitable chelating agents include disodium edetate, sodium citrate, condensed sodium phosphate and the like.

Examples of suitable antioxidants include sulfite, ascorbic acid, c-tocopherol, cysteine and the like.

Besides the above-mentioned, agar, casein, collagen and the like are pharmaceutically acceptable carriers.

When the agent for the prophylaxis and treatment of disturbance of visual function of the present invention is used in the form of an aqueous liquid, its pH is 4 to 9 in view of the stability of memantine and a salt thereof.

The oral preparations may be, for example, solid preparations (e.g., powders, granule, tablets and capsules) or liquid preparations (e.g., emulsions, syrups and suspensions).

Parenteral preparation includes, for example, injections, preparations for local administration to the eye, and the like. The injection includes subcutaneous injection, intravenous injection, intramuscular injection and the like. Injections may be aqueous or non-aqueous, and a solution or suspension.

The preparations for local administration to the eye include ophthalmic solution, ophthalmic ointment, gel and the like, with particular preference given to ophthalmic solution which may be aqueous or non-aqueous, and a solution or suspension.

The agent for the prophylaxis and treatment of disturbance of visual function of the present invention is preferably used as a preparation for local administration to the eye. More preferably, it is used as an ophthalmic solution, particularly an aqueous ophthalmic solution.

An aqueous injection can be prepared by, for example, dissolving memantine or a salt thereof in water for injection together with the above-mentioned preservatives, isotonizing agents, solubilizers and the like. An oily injection can be prepared by dissolving or suspending memantine or a salt thereof in propylene glycol, olive oil, sesame oil, cotton seed oil and the like.

An aqueous ophthalmic solution can be prepared by, for example, heating distilled water, dissolving a preservative therein, adding a solubilizer, and adding and completely dissolving compound >I! or a salt thereof. Where necessary, buffers, isotonizing agents, chelating agents, tackifiers and the like may be also added.

The aqueous ophthalmic suspension can be prepared by adding, besides the above-mentioned additives used for aqueous ophthalmic solutions, the aforementioned suspending agents as appropriate.

The pH of the above-mentioned aqueous ophthalmic solution and aqueous ophthalmic suspension is preferably 4 to 9, particularly preferably 5 to 8.

A non-aqueous ophthalmic solution can be prepared by dissolving or suspending memantine or a salt thereof in an aqueous solvent such as alcohols (e.g., ethanol, ethylene glycol, macrogol, propylene glycol, glycerol and the like) and an oily solvent such as fats and oils (e.g., olive oil, sesame oil, peanut oil, cotton seed oil, castor oil, corn oil and the like).

An ophthalmic ointment can be prepared by appropriately using, for example, petrolatum, plastibase, liquid paraffin and the like as a base.

An ophthalmic gel can be prepared by appropriately using, for example, carboxyvinyl polymer, polymer of ethylene maleic anhydride, polyoxyethylene-polyoxypropylene block copolymer, gellan gum and the like as a base.

While the dose of the agent for the prophylaxis and treatment of disturbance of visual function of the present invention varies depending on the administration route, kind of diseases, symptoms, age and body weight of patients, and the like, for example, it is preferably administered to an adult patient with asthenopia, axial myopia or retinal disease as an aqueous ophthalmic solution comprising memantine or a salt thereof, which is an active ingredient, in a concentration of 0.01 to 2.0 (W/V) %, preferably 0.1 to 1.0 (W/V) %, in a single dose of one to several drops thereof according to symptoms, once to several times a day, preferably 2 to 5 times a day, to one eye of a patient.

The present invention is described in more detail in the following by way of Examples, and the effects of the invention are clarified by way of Experimental Examples, which should not be construed as limiting the invention.

### EXAMPLE 1

This invention is especially useful in the use of memantine to normalize visual acuity deficits from outer retina (photoreceptor/RPE) injury. The discovery of the effect of memantine to normalize or improve visual acuity came from the observation that 4.5 months after transient IOP elevation in 2.5 kg rabbits pre-treated with an oral dose of 50 mg memantine or 50 mg sugar placebo, rabbits in the memantine group had normal visual acuities while rabbits in the placebo group had a reduction in acuity.

The results are shown in the FIGURE.

Histology (H&E) examination of eyes from the placebo group revealed a patchy loss of retinal cells that were predominantly outer retina in origin. Retinal cell degeneration was less in the memantine group compared with the placebo group.

### Protocol for Transient Elevation of IOP.

Rabbits were anesthetized with isofluorane, and prepared for unilateral acute retinal ischemia by raising IOP in the OD eye by 120 mm Hg for 45 minutes. To accomplish this, a reservoir with PBS was suspended 65 inches above the eye and connected to a 30 gauge needle inserted through the cornea into the anterior chamber. A drop of topical anesthetic (proparacaine) was placed upon the cornea prior to needle insertion.

### Protocol for Visual Acuity Measurement in Conscious Rabbits Using Sweep Visual Evoked Potential (swVEP).

swVEP is an electrophysiological technique for assessing visual acuity in young children who can't read the Snellen eye charts. Pattern reversal images of increasing spatial frequency are projected onto the macula while simultaneously recording electrical activity (VEP) from the scalp. Images with lower spatial frequency generate large signals which get smaller as the spatial frequency increases, until signal=noise; this threshold is the visual acuity. The procedure in rabbits involves first implanting permanent electrodes on the scalp to enhance signal strength and allow recording for the same position from follow-up visits. After two-weeks to allow for healing, acuity measurements can be made.

Rabbits were anesthetized with ketamine and xylazine for the implantation procedure. The scalp was aseptically prepared and implanted with four stainless steel screws (#0-80×⅜). Two active electrodes were placed at 6 mm on either side of the midline, 6 mm above bregma; 1 ground electrode was placed at midline, 6 mm above the active electrodes; and, 1 reference electrode was placed at midline, 6 mm above the grounding electrode.

For the acuity test, eyes were fully dilated with 1% tropicamide and 10% phenylephrine.

Rabbits were placed in specially-designed stainless steel restrainers that allowed projection of the pattern-reversal images unto the visual streak. Rabbits were fully-conscious. Images were projected via a specially-designed fundus camera stimulator under control of the PowerDiva software version 1.8.5. The animal's eye was located at 50 mm in front of camera which is equivalent to 50 cm from a 21 in CRT monitor. Recording electrodes were connected to Grass Neurodata Acquisition System (Model 12CA) with the following specifications:
Channel 1 for OD eye and Channel 2 for OS eye.
Filter range between 3 to 100 Hz.
Amplification: 50 K
Line frequency filter=OFF.

Vertical steady-state pattern-reversal sweep stimulus at spatial frequency range of 0.1 to 5 cycles per degree at a temporal frequency of 7.5 Hz were applied to the eye at mean luminance of 600 cd/m2 and contrast of 80%. Five to 40 trials, 10 secs each, were collected from each eye. The number of trials was based on the signal-to-noise ratio. Trials were averaged and the threshold (visual acuity) was determined by software or manual fitting at signal-to-noise ration no less than 2.5. Threshold values were then normalized by expressing as a percent of the contralateral eye.

### EXAMPLE 2

A group of patients who are diagnosed as qualified for experimental treatment are divided into two groups.

Group A, of 22 patients, is treated with a regimen in which they were administered a therapeutically effect amount of memantine in an oral dosage form may be a tablet comprising 5 or 10 mg memantine or a solution which comprises 2 mg/ml memantine.

A second group of 15 patients, Group B, is administered a placebo wherein the same solution is administered without memantine in a solution.

To evaluate the patients after treatment, visual acuity tests using standard Snellen eye charts were administered 3 months after treatment. The change in visual acuity is averaged for each group.

After 3 months, patients subjected to regimen A showe an improvement in visual acuity of +0.10 (an improvement of 1.0 would indicate an improvement of one line on the conventional Snellen eye charts). Patients subjected to regimen B decrease in visual acuity at an average of −0.40.

Thus, it appeared that regimen A wherein memantine is administered and was the better of these two protocols tested.

### EXAMPLE 3

### Time Course of Enhancement of Visual Acuity

Sixteen patients in the study are subjected to regimen A as described in Example 2 above and evaluated for visual acuity after 1 week and after 4 weeks as well as after 3 months. One week after treatment these patients have an average increase in visual acuity of +2.13; 4 weeks after treatment the average is +1.25 and after 3 months, +0.53.

What is claimed is:

1. A method for the treatment of disturbance of visual function which comprises administering a pharmaceutically effective amount of memantine to a patient in need of treatment of said disturbance.

2. The method according to claim 1, comprising local administration of memantine or a salt thereof to the eye.

3. The method according to claim 1, comprising administering memantine or a salt thereof in the form of a liquid preparation.

4. The method according to claim 3, comprising administering the memantine or a salt thereof in the form of an ophthalmic solution.

5. The method according to claim 4, wherein the ophthalmic solution is an aqueous ophthalmic solution.

6. The method according to claim 3, comprising administering memantine or a salt thereof in the form of an injection.

7. The method according to claim 5 comprising administering memantine or a salt thereof in the presence of a solubilizer.

8. The method according to claim 1 comprising systemically administering memantine or a salt thereof in the form of a solution.

9. The method according to claim 7 wherein said memantine or salt thereof is administered orally.

10. The method according to claim 1 comprising administering a pharmaceutical preparation containing memantine or a salt thereof in a concentration of 0.01-2.0 (W/V) %.

11. The method of claim 1 wherein said disturbance of visual function is a result of injury to the outer retina causing visual acuity deficits.

12. The method of claim 11 wherein said treatment results in an improvement in reading a Snellen Eye Chart of at least one line.
